(19) United States
(12) Patent Application Publication
(10) Pub. No.: US 2018/0243039 A1
(43) Pub. Date: Aug. 30, 2018

(54) SURGICAL SITE COVER COMPRISING CONFORMABLE POLYMERIC FILM AND METHODS OF USE

(71) Applicant: 3M INNOVATIVE PROPERTIES COMPANY, St. Paul, MN (US)

(72) Inventors: Elaine C. Ramires, São Paulo (BR); Jener De Oliveira, São Paulo (BR); Steven B. Heinecke, New Richmond, WI (US); Fernanda Martins, São Paulo (BR)

(21) Appl. No.: 15/754,715

(22) PCT Filed: Sep. 22, 2016
(86) PCT No.: PCT/US2016/053043

§ 371 Date: Feb. 23, 2018

Related U.S. Application Data
(60) Provisional application No. 62/234,997, filed on Sep. 30, 2015.

(51) Int. Cl. A61B 46/20; A61B 17/02

(52) U.S. Cl. CPC A61B 46/20; A61B 2046/205; A61B 17/0231

(57) ABSTRACT

A surgical site cover is described comprising an adhesive coated film. The adhesive coated film comprises a pressure sensitive skin contact adhesive layer disposed on a major surface of a conform able polymeric film layer. The adhesive coated film further comprises an opening or a peripheral edge of suitable size to expose a surgical site. A first end portion of at least one retraction member is permanently attached to the conform able polymeric film proximate the opening or peripheral edge and an opposing end portion of the retraction member comprises an attachment member. Also disclosed are kits and methods for applying the surgical site cover. In the preferred embodiment, the surgical site cover is a surgical eye cover, wherein the pressure sensitive skin contact adhesive isolates at least the eyelash line and at least the upper eyelid is retracted by means of applying a force to the retraction member.

### BACKGROUND

As described in U.S. Pat. No. 5,213,114, “surgical drapes are made in a wide variety of sizes and shapes for covering portions of the body. Drapes help maintain a sterile environment during surgery and help prevent foreign matter and organisms from entering the immediate vicinity of the surgery. In ophthalmologic examination and surgery, eye drapes are frequently used to surround and protect the eyeball. One type of drape has a central hole or opening, the drape material being used to cover the portions of the body surrounding the opening. Another type of drape, the incise drape, is a continuous piece of sheet material which is placed over the eye and an opening is cut to expose the eye and surround it with the drape material. Both types of drapes can effectively cover the exterior of the eyelids and surrounding skin but cannot readily cover the interior surfaces of the eyelids. The eyelids and eyelashes are particularly important to cover because they represent a potential source of bacteria and other contaminates. Additionally, stainless steel eyelid retractors, which pull the eyelids back from the eyeball, are used in many procedures, but it remains important to drape or cover the eyelids, even when retractors are used.”

### SUMMARY

In one embodiment, a surgical site cover is described comprising an adhesive coated film. The adhesive coated film comprises a pressure sensitive skin contact adhesive layer disposed on a major surface of a conformable polymeric film layer. The adhesive coated film further comprises an opening or a peripheral edge of suitable size to expose a surgical site. A first end portion of at least one retraction member is permanently attached to the conformable polymeric film proximate the opening or peripheral edge and an opposing end portion of the retraction member comprises an attachment member.

In typical embodiments, the opening is a slit or perforations that form a slit through the adhesive coated film. The retraction member is less conformable, has a lower elongation, and has a greater tensile strength than the conformable polymeric film. Prior to use, pressure sensitive adhesive layer(s) typically further comprise a removable release liner. Further, the surgical site cover typically further comprises a permanent or removable carrier frame at the periphery of the surgical site cover. The surgical site cover may comprise a one piece or multiple (e.g. two) piece construction. In one embodiment, the surgical site cover is a surgical eye cover.

The retraction member is typically permanently bonded to the adhesive coated film as packaged and received by a practitioner. However, in other embodiments, surgical site cover kits are described that comprise one or more adhesive coated films and separate retraction members for attachment to the adhesive coated film by the practitioner. In one embodiment, the adhesive coated film has a (e.g. centrally located) opening of suitable size to expose a surgical site. In another embodiment, the surgical site cover kit comprises at least two adhesive coated films having a peripheral edge of suitable size to border a surgical site.

Also described are methods of use of surgical site covers comprising providing the surgical site cover(s) or kits described herein and adhering the pressure sensitive skin contact adhesive layer to skin tissue such that the opening or peripheral edge is adjacent the surgical site.

The method typically comprises retracting skin tissue adjacent the surgical site by meaning of applying a force to at least one retraction member and maintaining the retraction by securing the attachment member of the retraction member. In some embodiments, the method is conducted in the absence of a speculum or other mechanical instrument utilized for the purpose of retracting the skin tissue adjacent the surgical site.

### BRIEF DESCRIPTION OF THE DRAWINGS

FIG. 1 is a cross-sectional view of an embodied surgical cover;

FIG. 2 is a cross-sectional view of an embodied surgical eye cover during an embodied use;

FIG. 3 is a top plan view of the surgical eye cover of FIG. 2 during an embodied use;

FIG. 3A is an enlarged top plan view of the eye area of the surgical cover of FIG. 3 during an embodied use;

FIG. 4 is a perspective view of an elliptical shaped surgical cover;

FIG. 5 is a perspective view of another embodied surgical cover;

FIG. 6 is a perspective plan view of a two-piece surgical cover;

FIG. 7 is a perspective plan view of another two-piece surgical cover;

FIG. 8 is a cross-sectional view of another embodied surgical cover;

FIG. 9 is a cross-sectional view of another embodied surgical cover.

### DETAILED DESCRIPTION

The present invention pertains to surgical cover articles and methods of use. Although the surgical cover is contemplated to be suitable for a variety of surgical procedures, in one embodiment, the surgical cover is a surgical eye cover.

With reference to the cross-sectional view of FIG. 1, one embodied surgical (e.g. eye) cover comprises an adhesive coated film comprising a pressure sensitive skin contact adhesive 116 disposed on a major surface 115 of a conformable polymeric film 114. The adhesive coated film further comprises a (e.g. centrally located) opening 125. A first end portion of at least one retraction member 130 is permanently attached (e.g. bonded) to the opposing major surface 113 of conformable polymeric film 114 proximate the opening 125.

The opening (125, 225, 325) may be a slit through the adhesive coated film as shown, a plurality of perforations, or an aperture. When the opening is an aperture, the adhesive coated film has been cut such that a portion of the adhesive coated film has been removed forming an open area. In contrast, when the opening is a slit or a plurality of perforations, the adhesive coated film has been cut, yet a portion of the adhesive coated film has not been removed. The opening has a suitable size and shape to expose a surgical site. The opening size and shape can be smaller than the surgical site since the size and shape of the opening can be adjusted by stretching the adhesive coated film. In one embodiment, the opening may be a slit or a plurality of perforations having a length about the same as an eyeball (e.g. about 30 mm+1-10 mm or about 30 mm+/−5 mm)

The retraction member 130 is typically a polymeric film or fibrous (e.g. nonwoven or cloth) material that is less conformable, has a greater stiffness, and is typically less stretchable at the force exerted during use than the conformable polymeric film 114, as will subsequently be described. A first end portion of the retraction member 130 is permanently attached to the conformable polymeric film 114 by any suitable mechanical or chemical means, such as heat sealing or ultrasonic sealing or adhesive bonding. By “permanently attached” or “permanently bonded” it is meant that the retraction member 130 remains bonded to the conformable polymeric film 114 while in use. Thus, in the case of surgical eye covers, the shear bond strength between the retraction member and conformable polymeric film is greater than the force exerted by closing ones eyelids (40 to 200 grams force/per eye lid of 30 mm (1.18 inches)=34 to 169 g/inch). In typical embodiments, the shear bond strength of the bond between the retraction member 130 and conformable polymer film 114 is at least 50, 125, 150, 175, 200 or 250 grams force per/inch (2.54 cm). In other embodiments, such shear bond strength is at least 300, 400, 500 or 1000 grams force per/inch. The retraction member material is also able to prevent an eyelid from closing and thus also meets and typically exceeds the shear bond strength just described. In typical embodiments, the retraction member typically does not elongate any more than 5, 4, 3, 2, or 1% with such applied force, but may elongate more at higher forces that would not be exerted by the eyelid. When a 3M™ Steri-Strip is utilized for the retraction member, the shear strength is typically at least 1000, 1500, 2000, 2500 or 3000 grams force/inch (grams force/2.54 cm). The shear bond strength properties can be modified for other types of surgical procedures by one of skill in the art.

The opposing end portion of the retraction member 130 comprises an attachment member suitable for attaching the opposing end portion of the retraction member to a surface a distance away from the surgical site. The attachment member (e.g. pressure sensitive adhesive) may be mated with a surface of the surgical eye cover, a nonwoven drape surface, a surface of the skin, a mechanical device such as a speculum etc. The attachment member can be a pressure sensitive adhesive or a mechanical fastener such as a snap or refastenable (e.g. hook and loop) material. In one embodiment, the retraction member 130 comprises a pressure sensitive adhesive layer 135 that is suitable for bonding to the conformable polymeric film 114 and also suitable for use as an attachment member for bonding the opposing end of the retraction member to another section of the conformable polymeric film 114 or to a portion of patient skin outside the periphery of the dressing, in order to maintain a retracted state of tissue surrounding the surgical site, such as the eyelids.

In some embodiments, such as depicted in FIG. 9, the retraction member 130 may comprise a non-adhesive tab 131 at the opposing end of the retraction member proximate the attachment member (e.g. pressure sensitive adhesive 135). The tab 131 provides a convenient and expedient means for a practitioner to grip the opposing end of the retraction member 130. The tab can be created by spacing the attachment member (e.g. pressure sensitive adhesive 135) a suitable distance from the end portion of the retraction member 130 such that the end portion does not include the attachment member, as shown in FIG. 9. Alternatively, a portion of the adhesive coated retraction member can be folded onto itself such that the outer layers comprise the retraction member material with the pressure sensitive adhesive disposed between. In yet another embodiment, a separate piece of a non-tacky material (e.g. paper, film, nonwoven) may be adhered to the end portion of pressure sensitive adhesive 135 to form the tab.

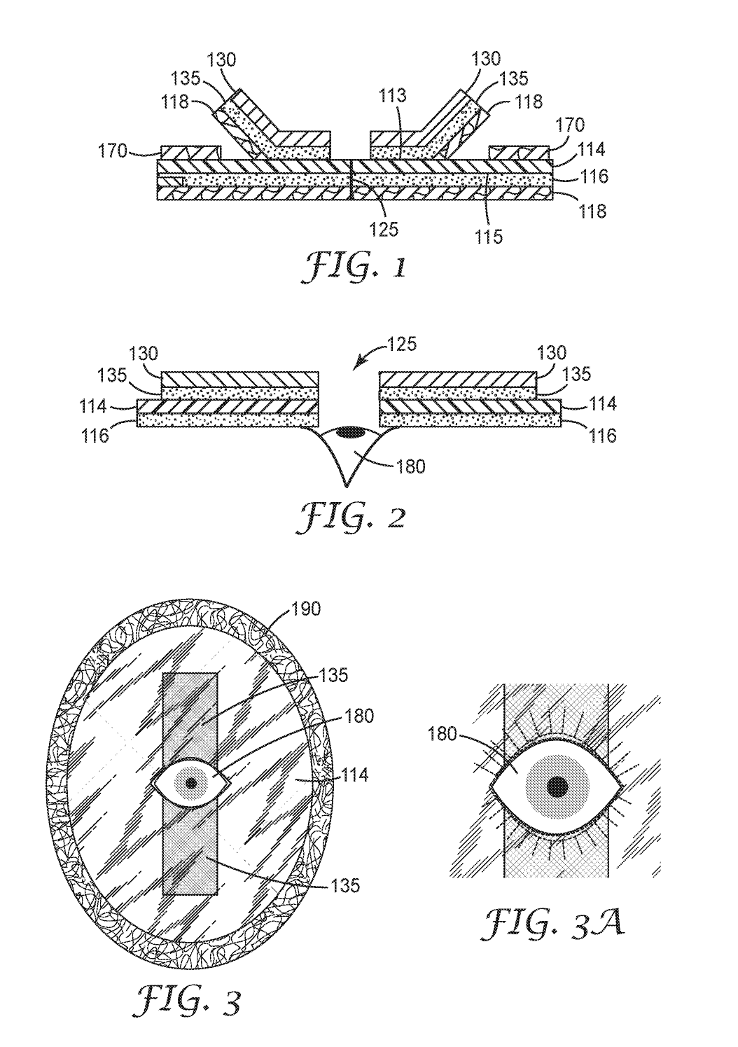

The surgical (e.g. eye) site cover typically further comprises one or more removable release liners 118 protecting the pressure-sensitive adhesive layers 116 and 135 until use. Various release liners are known such as those made of (e.g. kraft) papers, polyolefin films such as polyethylene and polypropylene, or polyester, or embossed films. The films are preferably coated with release agents such as fluorochemicals or silicones. For example, U.S. Pat. No. 4,472,480 describes low surface energy perfluorochemical liners. Examples of commercially available silicone coated release papers are supplied by LOPAREX (Willowbrook, Ill.) Other non-limiting examples of such release liners commercially available include siliconized polyethylene terephthalate films commercially available from H. P. Smith Co. and fluoropolymer coated polyester films commercially available from 3M under the brand “ScotchPak™” release liners. These release liners are removed at the time of use as depicted in FIG. 2.

The surgical (e.g. eye) site cover also typically further comprises a carrier frame 170 to facilitate the handling of the highly conformable polymeric film 114. Carrier frame 170 provides rigidity to the conformable polymeric film 114 after liner 118 is removed. The bond between the carrier 170 and the conformable polymeric film 114 is typically stronger than the bond between the adhesive 116 and the liner 118 such that the conformable polymeric film 114 remains attached to the carrier frame 170 when liner 118 is removed from the adhesive 116. The carrier frame may be removable carrier frame disposed on an outer surface of the conformable polymeric film such as described in U.S. Pat. No. 5,738,642; incorporated herein by reference. In other embodiments, the surgical (e.g. eye) cover may comprise a non-removable carrier frame such as a permanent (e.g. non-pressure sensitive) adhesive disposed on an outer or interior surface of the conformable polymeric film reinforcement such as described in U.S. Pat. No. 5,088,483; incorporated herein by reference. During use the removable carrier frame may optionally be removed after application of the pressure sensitive skin contact adhesive to the surgical site area as shown in FIG. 2. Alternatively, the removable carrier frame may remain bonded to the conformable film during the surgical procedure.

In one embodiment, the carrier frame 170 comprises an elastomeric nonwoven fabric/adhesive laminate based on melt blown webs of thermoplastic elastomeric small diameter fibers, or blown microfiber (BMF) webs. Elastomeric thermoplastic materials from which the microfiber webs can be prepared include, for example, elastomeric polyurethanes, elastomeric polyesters, elastomeric polyamides and elastomeric A-B-A block copolymers wherein A is a styrenic moieties and B is an elastomeric (e.g. isoprene or butylene) midblock. By use of an elastomeric nonwoven fabric/adhesive laminate carrier frame the adhesive coated film can provide sufficient rigidity while still allowing the adhesive coated film to stretch during removal or adjust the opening.

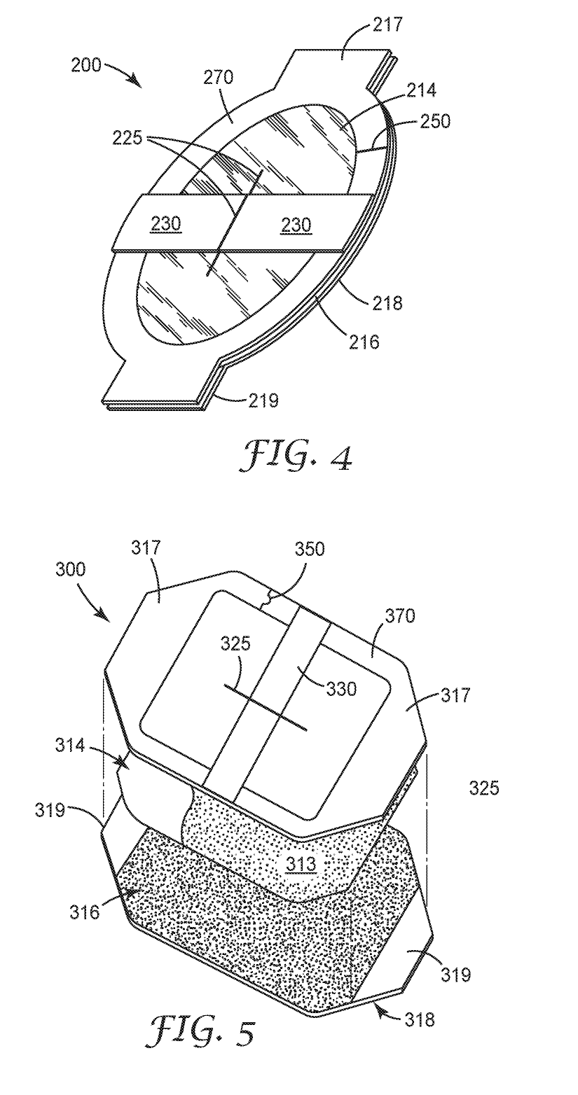

The exposed surface of the conformable polymeric film 314 may optionally comprise a low adhesion coating 313 as illustrated in FIG. 5. The low adhesion coating can reduce the surface friction of the exposed surface of the conformable polymeric film which can facilitate the use of the surgical eye cover with nonwoven surgical drapes covering other areas, such as the head, during the surgical procedure. When present, the low adhesion coating 313 is between the carrier frame 370 and the conformable polymeric film 314. The presence of the low adhesive coating 313 can insure that the carrier frame remains intact during the removal of release liner 318, yet the carrier frame can be subsequently be removed if desired. In one embodiment, the low adhesion coating 313 is present on the exposed surface area, but absent at the surface area wherein the retraction member 330 is bonded to the conformable polymeric film 314.

As shown in FIGS. 4-7, a (e.g. rectangular or oval) window portion is cut in the carrier material and removed creating the carrier frame (270, 370) and a window exposing a portion of the top face of the conformable polymeric film (214, 314). The carrier frame (270, 370) typically comprises a continuous periphery and optionally a cut (250, 350) that provides a location at which the carrier may be easily separated for removal of the carrier frame from the conformable polymeric film (214, 314).

Liner (218, 318) and carrier frame (270, 370) may both include tabs (217, 317 and 219, 319) which extend beyond the perimeter of conformable polymeric film (214, 314) to provide a means of applying the surgical eye cover without contacting the (sterile) pressure sensitive skin contact adhesive (216, 316). During use, liner (218, 318) is first removed from adhesive (216, 316) leaving the carrier frame (270, 370)/conformable polymeric film (214, 314)/pressure sensitive skin contact adhesive (216, 316) intact. The practitioner can manipulate the surgical (e.g. eye) cover (200, 300) using tabs (217, 317) on the carrier frame (270, 370) while viewing the surgical (e.g. eye) site area to which the cover (210, 310) will be attached through the window of conformable polymeric film (214, 314), which is preferably transparent or translucent.

With reference to FIGS. 2 and 3, the method of applying the surgical (e.g. eye) site cover generally comprises removing the release liner 118 to expose the pressure sensitive skin contact adhesive layer 116 that is disposed on the conformable polymeric film 114 and adhering the pressure sensitive adhesive skin contact adhesive layer 116 to a patent's skin tissue (e.g. facial area) such that the opening or peripheral edge is adjacent the surgical site. When the surgical site cover has a central opening, such as depicted in FIG. 1, the surgical cover is generally adhered such that the opening is centrally located relative to the surgical site (e.g. eye ball 180). The surgical cover(s) typically surround the surgical site.

The method further comprises retracting the skin tissue adjacent the surgical site (e.g. eyelids) by means of applying a force at least one retraction member. The retraction member is typically pulled in a direction parallel to the length of the retraction member and away from the surgical (e.g. eye) site. Once the skin tissue has been retracted, the method comprises maintaining the retraction of the skin tissue by securing (fixing or attaching) the attachment member such as by adhering pressure sensitive adhesive 135 to the conformable film 114 or other surface (e.g. nonwoven drape), or directly to the skin, at a location outside the periphery of the dressing. The method of retracting the skin tissue from a surgical site can be conducted in the absence of a speculum (e.g. in the case of eye lid tissue) or other mechanical instrument. However, practitioners may also utilize such instruments in combination with the surgical (e.g. eye) site cover described herein.

In one embodied application technique, the surgical cover may be first adhered at two opposing surfaces, a distance away from the surgical site, such as the forehead and cheekbone and/or nose and temple in the case of surgical eye drapes. The method may further comprise adjusting the size and/or shape of the surgical cover opening by stretching the adhesive coated film by means of the retraction member(s).

With reference to FIG. 3A, in the case of surgical eye covers, the method typically comprises adhering the pressure sensitive skin contact adhesive 116 to the eye area such that the adhesive 116 is in contact with and thereby isolates at the least the eyelash line from the surgical site. In one embodiment, the pressure sensitive skin contact adhesive 116 also isolates at least portions or the entire eye lid margin. Thus, as depicted in FIG. 3A, the waterline of the eye is isolated by the adhesive coated film of the surgical eye cover. At least the upper eyelid and typically both eyelids are retracted by means of applying force to the eyelids via the retraction members.

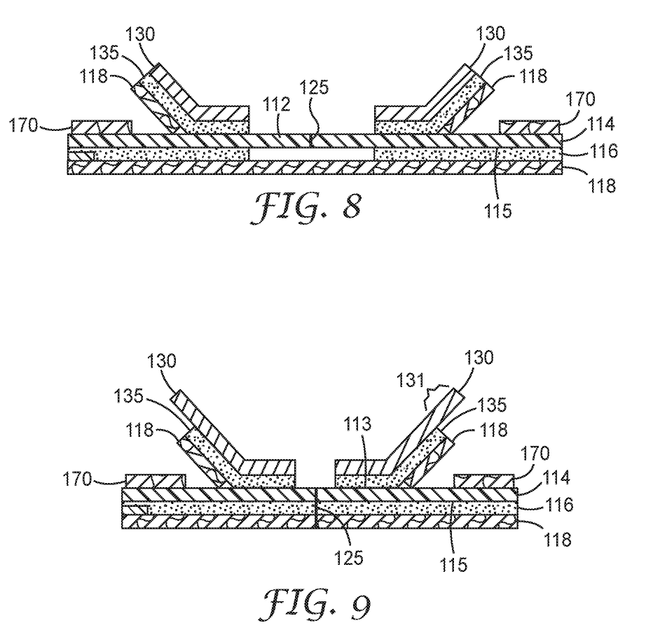

In an alternative embodiment, such as depicted in FIG. 8, the surgical (e.g. eye) cover comprises non-adhesive extensions 112, i.e. portions of conformable polymeric film adjacent the opening or edge that do not include the skin contact adhesive layer. These non-adhesive extensions can be tucked underneath the retracted skin tissue (e.g. eyelid) in order to isolate the surrounding tissue (e.g. eyelashes and eye lid margin) from the surgical site.

The size and shape of the adhesive coated film, opening and peripheral edge, can vary depending on the intended surgical site. In some embodiments, the surgical site cover has a dimension at least 1.5, 2, 2.5 or 3 times the length of the surgical site. Further, the surgical cover typically covers at least 2, 3, 4, or 5 cm of the skin tissue surrounding the surgical site.

With reference to FIG. 4, in one embodiment, the surgical (e.g. eye) site cover 200 may comprise an elliptical-shaped adhesive coated film comprising a conformable polymeric film layer 214 and a pressure sensitive skin contact adhesive 216 on a major surface of the conformable polymeric film 214. A first end portion of at least one retraction member 230 is permanently attached (e.g. bonded) to the conformable polymeric film 214 proximate opening 225.

In another embodiment, with reference to FIG. 5, the surgical (e.g. eye) site cover 300 may comprise a rectangular-shaped adhesive coated film comprising a conformable polymeric film layer 314 and a pressure sensitive skin contact adhesive 316 on a major surface of the conformable polymeric film 314. A first end of at least one retraction member 330 is permanently bonded to the conformable polymeric film 314 proximate opening 325.

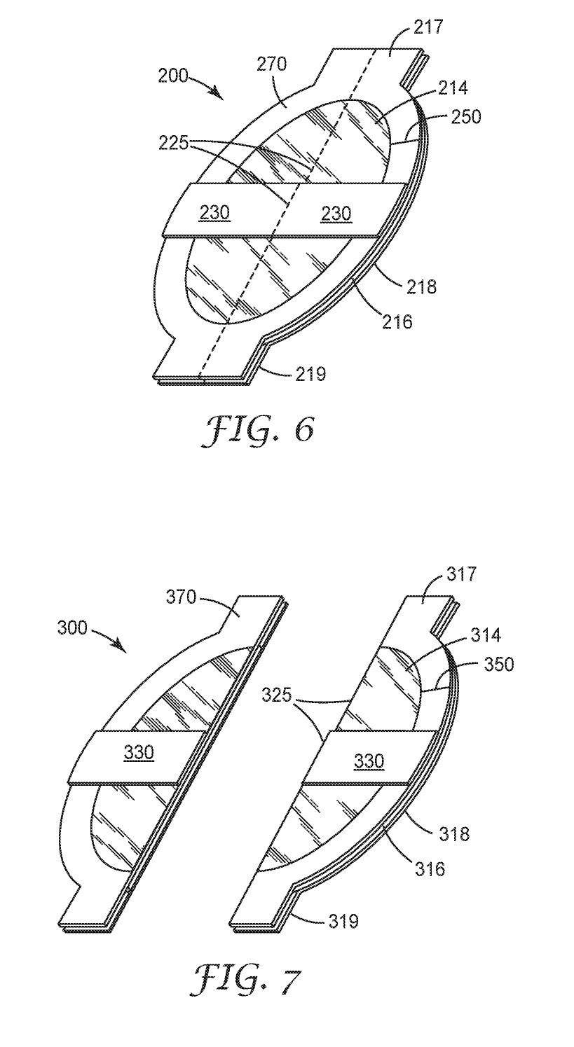

In some embodiments, the surgical (e.g. eye) site cover has a single (e.g. centrally located) opening such as illustrated in FIGS. 1-5. In another embodiment, the surgical (e.g. eye) site cover may comprise two or more pieces of adhesive coated film. In one embodiment, illustrated in FIG. 6, the surgical (e.g. eye) site cover may be scored as shown in FIG. 6, wherein the surgical (e.g. eye) site cover is packaged as a single piece that can be separated into two pieces at the time of use. Alternatively, the surgical (e.g. eye) site cover may be packaged as two separate pieces as shown in FIG. 7. In this embodiment, at least one piece comprises at least one retraction member 330 permanently attached (e.g. bonded) to a major surface of the conformable polymer films 314 proximate a peripheral edge of the surgical (e.g. eye) site cover.

The surgical site cover preferably comprises the adhesive coated film and at least one retraction member preassembled as depicted in FIGS. 1-8. However, in other embodiments, surgical cover kits are described that comprise one or more pieces of the adhesive coated film and one or more retraction members. The retraction members comprise a first end portion comprising a first attachment member for attachment to the adhesive coated film and a second attachment member at an opposing end portion. In some embodiments, the adhesive coated film and retraction member may be contained in the same package. In one embodiment, the adhesive coated film of the surgical site cover kit comprises a (e.g. centrally located) opening of suitable size to expose a surgical site. In another embodiment, the surgical site cover comprises at least two adhesive coated films having a peripheral edge of suitable size to border a surgical site.

The conformable polymeric film generally acts as a barrier to bacteria as well as liquid water or other liquids.

The polymeric film is conformable to anatomical surfaces. As such, when the surgical (e.g. eye) cover is applied to an anatomical surface, it conforms to the surface. A measure of conformability is the “F” value which provides a measure of force necessary to extend the polymeric film. F₁₀ value, as referred to herein, is determined using ASTM test method D 3759, except that the force measurements are taken at ten percent elongation. The cross-head speed is set at ten inches per minute and the chart speed is set at ten inches (25.4 cm) per minute. The gauge length is set at two inches (5.1 cm) with the test sample cut to test a one-inch width (2.54 cm). The F₁₀ value gives an approximation of the motion of the body surface and ability of a material to stretch with these body deformations. The F₁₀ value of the conformable polymeric film is no greater than about 1000 grams force per one-inch width (394 g/cm) and preferably no greater than about 750 (295 g/cm) or about 500 grams force per one-inch width (197 g/cm). In some embodiments, the F₁₀ value is less than 450, 400, 350, 300, 250, 200 or 150 grams force per one-inch width. In some embodiments, the F₁₀ value is at least 25, 30, 35, 40, or 50 grams force per one-inch width. The (e.g. adhesive coated) conformable polymeric film typically has a similar F₁₀ in both machine direction and cross-web direction.

Another property that relates to conformability is “Handle-O-Meter” data. The “Handle-O-Meter” data measures conformability of materials by measuring effects of forces perpendicular to the material. This measurement can be made in accordance with a modified INDA Standard Test for Handle-O-Meter stiffness, IST 90.0-75 (R82) or TAPPI T498 Su-66 modified in that 2.5 cm by 7.6 cm samples were measured and actual readings were taken from a 200 gram scale. The adhesive coated film surfaces of the samples can be coated with talc powder and were placed in the instrument adhesive side up. Readings were taken in grams-force units and were taken in both the machine and cross directions. In some embodiments, the Handle-O-Meter of the conformable polymeric film is no greater than 5, 4.5, 4, 3.5, 3, 2, 1.5 or 1 grams-force per inch (2.54 cm). In some embodiments, the Handle-O-Meter value is at least 0.1, 0.15, 0.2 grams force per inch (2.54 cm) width. The Handle-O-Meter value of the (e.g. adhesive coated) film is typically similar in both machine direction and cross-web direction.

The (e.g. adhesive coated) conformable polymeric film typically has a relatively low tensile strength as compared to the retraction member. For example, the tensile strength at break of the (e.g. adhesive coated) conformable polymeric film is typically no greater than about 5 kgf/inch, and in some embodiments no greater than 4.5, 4, 3.5, or 3 kgf/inch. The elongation at break of the (e.g. adhesive coated) conformable polymeric film is typically at least 100%, 150% or 200% ranging up to 500%, 600%, 700%, 800%, 900% or 1000%. At low elongations such as 5%, 10%, 15%, or 20%, the (e.g. adhesive coated) conformable polymeric film typically exhibits a tensile strength of no greater than 1 kgf/inch and in some embodiments no greater than 0.9, 0.8, 0.7, 0.6, 0.5, 0.4, 0.3, or 0.2 kgf/inch. The tensile strength and elongation properties of the (e.g. adhesive coated) conformable polymeric film as described herein are determined according to the test method described in the forthcoming examples. In typical embodiments, the tensile strength and elongation properties of the conformable polymeric film is substantially the same as the adhesive coated conformable polymeric film.

In some embodiments, the conformable polymeric film is sufficiently conformable such that it is conformable to animal anatomical parts or joints. When the joint is flexed and then returned to its unflexed position, the conformable polymeric film stretches to accommodate the flexion of the joint, but is resilient enough to continue to conform to the joint when the joint is returned to its unflexed condition. Although the conformable polymeric film can conform to an anatomical surface even when the surface is moved, movement (such as closing of the eyelids) is restricted by the retraction member during use. A description of suitable conformable polymeric films can be found in issued U.S. Pat. Nos. 5,088,483 and 5,160,315; incorporated herein by reference. Preferred conformable polymeric films are elastomeric polyurethane, co-polyester, or polyether block amide films. These films combine the desirable properties of resiliency, high moisture vapor permeability, and transparency found in preferred backing layers. Examples include, but not limited to: polyurethanes commercially available from B.F. Goodrich, Cleveland, Ohio, under the trade designation ESTANE, including ESTANE 58237 and ESTANE 58245; polyetheramide block copolymers commercially available from Elf Atochem, Philadelphia, Pa., under the trade designation PEBAX, including PEBAX MV 1074; polyether-ester block copolymers commercially available from DuPont, Wilmington, Del., under the trade designation HYTREL; and thermoplastic elastomers commercially available from DSM Engineering Plastics, Evansville, Ind., under the trade designation ARNITEL VT.

The thickness of the conformable polymeric film can vary. In typical embodiments, the thickness is at least about 10 or 15 μm and typically no greater than about 250 μm. In some embodiments, the thickness of the conformable polymeric film is no greater than 200, 150, 100, or 50 μm.

In some embodiments, the conformable polymeric film is a high moisture vapor permeable film, such as described in U.S. Pat. No. 3,645,835; incorporated herein by reference. The adhesive coated film may permit transmission of moisture vapor at a rate equal to or greater than human skin. Preferably, the uncoated film (i.e., without the skin contact adhesive) transmits moisture vapor at a rate of at least 10 g/10 cm²/24 hrs, 15 g/10 cm²/24 hrs, or 20 g/10 cm²/24 hrs as measured by EN-13726-1:2002 Section 3.3. The adhesive coated film may transmit moisture vapor at rate of at least 0.8 g/10 cm²/24 hours, or greater than 1.6 g/10 cm²/24 hours. However, moisture vapor permeability may be of less importance for surgical site covers that are used for short durations of time.

The surgical (e.g. eye) cover further comprises a skin contact pressure sensitive adhesive (PSA) layer. Examples of PSAs include rubber based adhesives (e.g., tackified natural rubbers, synthetic rubbers, and styrene block copolymers), acrylics (e.g., polymerized (meth)acrylates), poly(alpha-olefins), polyurethanes, and silicones. Amine containing polymers can also be used which have amine groups in the backbone, pendant thereof, or combinations thereof. A suitable example includes a poly(ethyleneimine).

Useful adhesives can be any of those that are compatible with skin and useful for wound dressings, such as those disclosed in U.S. Pat. No. Re. 24,906 (Ulrich), U.S. Pat. No. 5,849,325 (Heinecke et al.), and U.S. Pat. No. 4,871,812 (Lucast et. al.) (water-based and solvent-based adhesives); U.S. Pat. No. 4,833,179 (Young et al.) (hot-melt adhesives); U.S. Pat. No. 5,908,693 (Delgado et al.) (microsphere adhesives); U.S. Pat. Nos. 6,171,985 and 6,083,856 (both to Joseph et al.) (low trauma fibrous adhesives); and, U.S. Pat. No. 6,198,016 (Lucast et al.), U.S. Pat. No. 6,518,343 (Lucast et al.), and U.S. Pat. No. 6,441,092 (Gieselman) (wet-skin adhesives). Inclusion of medicaments or antimicrobial agents in the adhesive is also contemplated, as described in U.S. Pat. No. 4,310,509 (Berglund) and U.S. Pat. No. 4,323,557 (Rosso).

Silicone and acrylic based pressure sensitive adhesives are most commonly utilized for adhering to the skin.

Silicone PSAs typically include two major components, a polymer or gum, and a tackifying resin. The polymer is typically a high molecular weight polydimethylsiloxane or polydimethyl-diphenylsiloxane, that contains residual silanol functionality (SiOH) on the ends of the polymer chain, or a block copolymer including polydiorganosiloxane soft segments and urea terminated hard segments. The tackifying resin is generally a three-dimensional silicate structure that is endcapped with trimethylsiloxy groups (OSiMe₃) and also contains some residual silanol functionality. Examples of tackifying resins include SR 545, from General Electric Co., Silicone Resins Division, Waterford, N.Y., and MQD-32-2 from Shin-Etsu Silicones of America, Inc., Torrance, Calif. Manufacture of typical silicone PSAs is described in U.S. Pat. No. 2,736,721 (Dexter). Manufacture of silicone urea block copolymer PSA is described in U.S. Pat. No. 5,214,119 (Leir et al.).

Acrylic adhesive typically comprise a copolymer of at least one C₄-C₁₂ alkyl (meth)acrylate such as isooctyl acrylate or 2-ethylehexylacrylate and at least one high Tg (e.g. polar) comonomer such as (meth)acrylamide, N-vinyl pyrrolidone, poly(ethylene oxide)acrylate, and mixture thereof. In typical embodiments, the acrylic adhesive comprises at least 90 wt.-% C₄-C₁₂ alkyl (meth)acrylate(s). Suitable examples include a 90:10 isooctyl acrylate:acrylic acid copolymer, a 70:15:15 isooctyl acrylate:ethylene oxide acrylate:acrylic acid terpolymer, and a 25:69:6 2-ethylhexylacrylate:butyl acrylate:acrylic acid terpolymer. Another acrylic adhesive composition includes a 97:3 isooctyl acrylate:acrylamide copolymer 65:15:20 2-ethylhexylacrylate:acrylic acid:copolymer blended with a nonreactive polyalkylene oxide copolymer under the trade designation PLURONIC. Additional useful adhesives are described in U.S. Pat. Nos. 3,389,827, 4,112,213, 4,310,509, and 4,323,557.

In some embodiments, the silicone adhesive may be characterized as gentle to skin such as described in U.S. Pat. No. 8,541,481, US2013/0040073, U.S. Pat. No. 7,407,709 and U.S. Pat. No. 7,807,268. Although more aggressive pressure sensitive skin contact adhesives may be used for other surgical sites, for surgical eye covers, the adhesive has a relatively low initial peel value and relatively low peel value at 48 hours. Although such surgical site covers are generally used for much sorter durations of time than 48 hours, the 48 hour peel value is indicative of the build in adhesion as a function of time. The initial 90 degree peel adhesion to skin, as measured according to the skin adhesion test method described in U.S. Pat. No. 5,088,483 is typically no greater than about 3, 2.5, 2, 1.5, or 1 N/100 mm.

The peel adhesion to skin is sufficient to maintain the bond strength between the pressure sensitive skin contact adhesive and the skin tissue adjacent the surgical site, especially during retraction of the skin tissue and while maintaining retraction. Further, the peel adhesion to skin after a dwell time of 48 hours is typically no greater than about 10, 9, 8, 7, 6 or 5 N/100 mm. The peel adhesion to skin of the surgical eye drape is preferably substantially lower than standard incise drapes.

The adhesive coated conformable polymeric film can stretch release from the eyelid with a low amount of shear force. The maximum stretch release strength of the adhesive coated conformable polymeric film is typically no greater than 2.0, 1.9, 1.8, 1.7, 1.6, 1.5, 1.4 kgf/inch (2.54 cm) and in some embodiments at least 0.2, 0.3, 0.4, 0.5, 0.6, 0.7, 0.8, 0.9 or 1 kgf/inch. The percent elongation at the maximum stretch release strength is typically at least 50% and no greater than about 600%. In some embodiments, the percent elongation at the maximum stretch release strength is less than 500%, 450%, 400%, or 350%. The stretch release strength at 60%, 120%, 180%, or 240% is typically no greater than 1.34 kgf/inch and in some embodiments, no greater than 1.2, 1.1, 1.0, 0.9, 0.8, 0.7, 0.6, or 0.5 kgf/inch. The stretch release properties of the adhesive coated conformable polymeric film as described herein are determined according to the test method described in the forthcoming examples.

The pressure sensitive adhesive can be coated onto the conformable polymeric film by a variety of processes, including, direct coating, lamination, spray coating, and hot lamination. In some embodiments, the pressures sensitive adhesive may be coated as a microstructured adhesive layer.

The skin contact adhesive is typically present as a continuous adhesive layer at least at the locations adjacent the opening or peripheral edge of the surgical cover in order that the adhesive forms a seal surrounding the surgical site. The skin contact pressure sensitive adhesive also typically has low absorbency (i.e., less than 25% its dry weight, and preferably less than 10% its dry weight) when submerged in isotonic saline at 37° C. for 24 hours such that bodily fluid do not disrupt the seal formed by the adhesive. Other areas of the adhesive coated film (e.g. central portion of eye lid) may comprise a discontinuous skin contact pressure sensitive adhesive such as can be obtained by pattern coating. This approach can reduce the overall peel adhesion of the surgical (e.g. eye) cover from the skin during removal.

In one embodiment, the surgical (e.g. eye) cover is formed by bonding one or more retraction members to transparent or translucent wound dressing articles such as are available under the trade designation TEGADERM, available from 3M Co., St. Paul, Minn.

The retraction members can have various size and shapes. In typical embodiments, the retraction members have an elongated shape, meaning having a greater length than its width. In some embodiments, the retraction members are rectangular. The retraction members may terminate at a peripheral edge of the adhesive coated film or may have a length longer than the adhesive coated film.

The retraction member typically comprises a polymeric film or (e.g. nonwoven or woven) fibrous material that is less conformable than the conformable polymeric film. For example, the F₁₀ value of the retraction member is greater than the conformable polymeric film in the direction the retraction member is pulled during use (which is typically is a direction parallel to the length of the retraction member). In some embodiment, the F₁₀ value of the retraction member is at least 50%, 100%, 200%, 250%, 500%, or 1000%+ greater than the conformable polymeric film. In some embodiments, the F₁₀ value of the retraction member is at least 500, 1000, 2000, 2500, 5000, 6000, 6500, or 7000 grams force per one-inch width.

Further, the retraction member typically comprises a polymeric film or (e.g. nonwoven or woven) fibrous material that has a higher Handle-O-Meter value than the conformable polymeric film. In some embodiments, the Handle-O-Meter of the retraction member is greater than 2, 3, 4, 5 grams-force per inch (2.54 cm) and in some embodiments at least 10, 15, 20, 25, 30, or 35 grams-force per inch (2.54 cm) in the direction the retraction member is pulled during use (e.g. parallel to the length of the retraction member).

The tensile strength of the retraction member (e.g. material) is greater than the force exerted by a closing eyelid as previously described. In some embodiments, the tensile strength at break of the retraction member (e.g. material) is greater than the tensile strength at break of the (e.g. adhesive coated) conformable polymeric film and is typically at least 3 kgf/inch. In some embodiments, the tensile strength of the retraction member (e.g. material) is at least 4, 5, 6, 7, 8, 9, or 10 kgf/inch and typically does not exceed 20 kgf/inch.

The retraction member (e.g. material) typically has a low elongation as compared to the conformable polymeric film. The maximum elongation can ranges from about 5 to 50%. The retraction member (e.g. material) also typically exhibits a relatively high tensile strength at low elongations. For example, the tensile strength may be at least about 2, 3, 4, 5 kgf/inch at 5% elongation and range up to about 6, 7, 8, 9, or 10 for elongations of 10%, 15%, or 20%.

In one embodiments, the retraction member is a (e.g. polyester) nonwoven material such 3M™ Spunlaced Polyester Nonwoven Medical Tape 1776. The nonwoven of the retraction member may comprise fiber reinforcement such as in the case of 3M™ Steri-Strip Skin Closure 1548. In another embodiment, the retraction member is a (e.g. rayon acetate or cotton) woven cloth material such 3M™ Rayon Acetate Woven Medical Tape 1538 or 3M™ Cloth Adhesive Tape 2950. In another embodiments, the retraction member is a microporous (e.g. rayon) nonwoven such as 3M™ MICROPORE 1530 Surgical Tape.

In some embodiments, the surgical eye cover may further comprise an absorbent layer or absorbent members disposed on a portion of the conformable polymeric film. For example, the surgical (e.g. eye) cover may further comprise a nonwoven drape 190 bonded to the periphery of the surgical site cover. The nonwoven drape may further comprise a fluid collection pouch A surgical (e.g. eye) cover is typically provided in a package format (i.e., positioned in a sealed package). The interior of the sealed package is typically sterile. The packaging material may be, for example, a polymeric package such as polyethylene, polypropylene, copolymers of ethylene and propylene, polybutadiene, ethylene-vinyl acetate, ethylene-acrylic acid, or ionomeric films, or special papers and Tyvek®. Suitable foil packages can include aluminum foil packages. In some embodiments, the packaging material may be used as sheets of material which are placed above and below the surgical (e.g. eye) cover and then sealed on four sides to generate the package. In other embodiments, a pre-made pouch is utilized which has 3 sides already sealed. After the surgical (e.g. eye) cover is placed within the pouch the fourth side is sealed to form the package. Sealing of the package can be achieved by heat sealing (i.e. by the application of heat and pressure to form a seal) or the use of adhesive sealants can be used to seal the packages (for example pressure sensitive adhesive sealants or cold seal sealants). Typically, heat sealing is used. Additionally, packaging systems can be used which include a porous package that is then placed in a non-porous package, such as a foil package. The foil package prevents moisture loss prior to use and the porous package permits easy handling during use.

### EXAMPLES

### Materials

Materials utilized in the Examples are shown in Tables 1 and 2.

TABLE 1
Examples 1-4: Adhesive Coated Conformable Polymeric Films

| | Film | | Adhesive | |
|---|---|---|---|---|
| Ex- | Backing | | Layer | |
| am- | Approx. | Film Backing | Approx. | Adhesive layer |
| ple | Thickness | Material | thickness | Composition |
| Ex. 1 | 0.8 mil | Polyurethane | 1 mil | An acrylate |
| | (20 μm) | film | (25 μm) | PSA described in |
| | | backing | | Example 2 of US6461467 |
| Ex. 2 | 1 mil | Elastomeric | 1.7 mil | An acrylate PSA similar |
| | (25 μm) | copolyester | (43 μm) | to Example 2 of |
| | | (PET) | | US6461467 with |
| | | backing | | a 90/10 weight |
| | | | | ratio of isooctyl |
| | | | | acrylate/acrylamide |
| | | | | copolymer |
| Ex. 3 | 1.7 mil | Low density | 0.9 mil | An acrylate PSA similar |
| | (43 μm) | polyethylene | (23 μm) | to Example 2 of |
| | | backing | | US6461467 with |
| | | | | a 90/10 weight |
| | | | | ratio of isooctyl |
| | | | | acrylate/acrylamide |
| | | | | copolymer |
| Ex. 4 | 1 mil | Block | 2 mil | An acrylate PSA |
| | (25 μm) | copolymer of | (51 μm) | similar to |
| | | polytetra- | | Example 2 of |
| | | methylene | | US6461467 with |
| | | ether with poly | | a 91/9 weight ratio |
| | | (tetrame- | | of isooctyl |
| | | thyleneterephtha | | acrylate/acrylamide |
| | | late) | | copolymer |
| | | | | containing |
| | | | | povidone-iodine |
| | | | | antimicrobial agent |

TABLE 2
Examples 5-9 Retraction Member Materials

| Exam- | Material Description | Source |
|---|---|---|
| ple | | |
| Ex. 5 | 3M ™ Spunlaced Polyester Nonwoven Medical | 3M Company, |
| | Tape 1776: polyester backing, coated with a | St. Paul, MN |
| | medical, pressure sensitive acrylate adhesive | |
| Ex. 6 | 3M ™ Rayon Acetate Woven Medical Tape 1538; | 3M Company, |
| | Rayon acetate woven cloth backing coated with a | St. Paul, MN |
| | medical, pressure sensitive acrylate adhesive | |
| Ex. 7 | 3M ™ MICROPORE 1530 Surgical Tape; | 3M Company, |
| | microporous rayon nonwoven backing coated with | St. Paul, MN |
| | a medical, pressure sensitive acrylate adhesive | |
| Ex. 8 | 3M ™ Cloth Adhesive Tape 2950; high | 3M Company, |
| | strength cotton backing coated with a | St. Paul, MN |
| | medical, pressure sensitive acrylate | |
| | adhesive | |
| Ex. 9 | 3M ™ Steri-Strip Skin Closure 1548; nonwoven | 3M Company, |
| | backing, fiber reinforced with a medical, pressure | St. Paul, MN |
| | sensitive acrylate adhesive | |

### Test Methods

### Tensile Test Method

Percent elongation was measured using a Universal test machine available from Kratos Industrial Equipment Ltda., BR, model K2000MP with a load cell of 20 kgf (196 N), depending on the properties of the backing to be tested, and with the gauge distance and the kart speed set according to the backing characteristics, as set forth in Table 3 below. The sample size was between 20 and 100 mm (as described in table 3) length by 25.4 mm width.

### Stretch Release Test Method

Testing was based on the ASTM D3330M-90 except that peel was performed at 0 degree instead of 180 degrees. This was to simulate and evaluate the stretch release removal process. Also, each example test material was sized to 50 mm length by 19 mm width and was affixed to a substrate of polyurethane elastic foam instead of stainless steel test surface. The polyurethane elastic foam simulated human skin better than stainless steel during the stretch release removal of the example materials. The polyurethane elastic foam was the same material as the foam pad used in the 3M TEGADERM HIGH PERFORMANCE FOAM ADHESIVE DRESSING, Medium Oval, Pad size 4 inch×4½ inch (10 cm×11 cm), product number 90613, available from 3M Company of St. Paul, Minn. The polyurethane elastic foam was adhered to the stainless steel instrument surface using a double sided adhesive tape 3M™ Transparent Polyethylene, Double Coated Tape 9889, available from 3M Medical Materials and Technologies, St. Paul, Minn. The “skin” facing side of the polyurethane elastic foam was used as the test surface; the opposite side was adhered to the stainless steel instrument surface using the 9889 double sided tape.

TABLE 3
Gauge Distance and Test Speed for Elongation Testing

| Conditions | Distance between gauges | Test speed |
|---|---|---|
| <100% Elongation | 100 mm | 100 mm/min |
| between 100-400% | 50 mm | 200 mm/min |
| >400% | 20 mm | 200 mm/min |

### Test Results

### Tensile Strength, Percent Elongation, and Stretch Release

Examples were tested according to the Tensile Test Method to determine the Tensile Strength in kilogram-force/inch (kgf/inch) and Percent Elongation at break (%). Examples 1-4 represent relatively elastic backings having a percent elongation of at least 100% that can be used as the conformable polymeric film of the present disclosure. Examples 5-9 represent relatively non-elastic materials having a percent elongation of less than 100% that can be used as the retraction member material of the present disclosure. Test results are summarized in Tables 4-6.

TABLE 4
Tensile Strength for Adhesive Coated Conformable Polymeric
Film (Ex. 1-4) and Retraction Member (Ex. 9)

| | | | | | | Tensile |
|---|---|---|---|---|---|---|
| | | | Tensile | Tensile | Tensile | Strength |
| | Tensile | | Strength | Strength | Strength | at |
| | Strength | Percent | at | at 10% | at 15% | 20% |
| | at | Elon- | 5% | Elon- | Elon- | Elon- |
| | break | gation | Elongation | gation | gation | gation |
| Ex. | kgf/inch | Maximum | kgf/inch | kgf/inch | kgf/inch | kgf/inch |
| Ex. 1 | 3.31 | 586.49 | 0.17 | 0.21 | 0.25 | 0.28 |
| Ex. 2 | 2.62 | 717.65 | 0.34 | 0.42 | 0.47 | 0.52 |
| Ex. 3 | 1.86 | 229.46 | 0.53 | 0.65 | 0.73 | 0.80 |
| Ex. 4 | 3.07 | 835.55 | 0.32 | 0.39 | 0.45 | 0.48 |
| Ex. 9 | 10.33 | 35.40 | 4.73 | 6.20 | 7.32 | 8.46 |

Results are an average of 10 replicates for Examples 1-4, and 9.

TABLE 5
Stretch Release of Adhesive Coated Conformable Polymeric Film

| | | | Strength | Strength | Strength | Strength |
|---|---|---|---|---|---|---|
| | | Percent | at | at | at | at |
| | | Elon- | elon- | elon- | elon- | elon- |
| | | gation | gation | gation | gation | gation |
| | Maxi- | Maxi- | of | of | of | of |
| | mum | mum | 30 mm | 60 mm | 90 mm | 120 mm |
| | strength | at total | (60%) | (120%) | (180%) | (240%) |
| Ex. | (kgf/inch) | release | (kgf/inch | (kgf/inch) | (kgf/inch) | (kgf/inch) |
| Ex. 1 | 1.06 | 319.70 | 0.53 | 0.61 | 0.66 | 0.77 |
| Ex. 2 | 1.32 | 493.28 | 0.81 | 0.85 | 0.88 | 0.92 |
| Ex. 3 | 1.91 | 67.65 | 1.29 | 0.00 | 0.00 | 0.00 |
| Ex. 4 | 1.53 | 533.05 | 0.76 | 0.85 | 0.93 | 0.97 |

Results are an average of 10 replicates for Examples 1, 3, and 4. Example 2 was an average of 9.

TABLE 6
Tensile Strength at Break & Percent Elongation
Maximum for Retraction Member Materials

| Exam- | Tensile Strength at break | Percent Elongation |
|---|---|---|
| ple | kgf/inch (N/inch) | Maximum |
| Ex. 5 | 8.29 (81.3 ) | 41.58 |
| Ex. 6 | 16.54 (162.2) | 21.20 |
| Ex. 7 | 3.58 (35.1) | 13.02 |
| Ex. 8 | 13.65 (133.9) | 5.90 |
| Ex. 9 | 10.33 (101.3) | 35.40 |

Results are an average of 10 replicates for Examples 5-7, and 9. Example 8 was an average of 9.

1. A surgical site cover comprising:
an adhesive coated film comprising
a pressure sensitive skin contact adhesive layer disposed on a major surface of a conformable polymeric film layer;
wherein the adhesive coated film comprises an opening or a peripheral edge of suitable size to expose a surgical site and a first end portion of at least one retraction member is permanently attached to the conformable polymeric film proximate the opening or peripheral edge of the opening, and an opposing end portion of the retraction member comprises an attachment member;
wherein the conformable polymeric film is characterized by any one or combination of the following properties:
i) a F₁₀ value of less than 1000 grams force/inch;
ii) a Handle-O-Meter value less than 5 grams force/inch;
iii) a maximum elongation greater than 200%; and
iv) a tensile strength of no greater than 1 kgf/inch (2.54 cm) for an elongation of 5% or 10%.

2. The surgical site cover of claim 1 wherein the opening is a slit or perforations that form a slit through the adhesive coated film.

3. (canceled)

4. The surgical site cover of claim 1 wherein the retraction member is characterized by any one or combination of the following properties:
i) a F₁₀ greater than the conformable polymeric film;
ii) a Handle-O-Meter value greater than the conformable polymeric film;
iii) a maximum elongation less than 100%; and
iv) a tensile strength of at least 2 kgf/inch for an elongation of 5% or 10%.

5. The surgical site cover of claim 1 wherein the retraction member comprises a strip of a polymeric film or fibrous material or a combination thereof optionally including reinforcement with fibers.

6. The surgical site cover of claim 1 comprising at least one retraction member on each opposing side of the opening.

7. The surgical site cover of claim 1 comprising at least two retraction members on the same side of the opening or the same peripheral edge.

8. The surgical site cover of claim 1 wherein the attachment member is a pressure sensitive adhesive or mechanical fastener.

9. The surgical site cover of claim 1 further comprising a non-adhesive tab at the end of the retraction member proximate the attachment member.

10. The surgical site cover of claim 1 further comprising a removable release liner in contact with the pressure sensitive skin contact adhesive layer.

11. The surgical site cover of claim 1 further comprising a permanent or removable carrier frame at the periphery of the surgical site cover.

12. The surgical site cover of claim 1 wherein the surgical eye cover further comprises a nonwoven drape bonded to the periphery of the surgical site cover.

13. The surgical site cover of claim 1 wherein the surgical site cover is free of non-adherent extensions suitable for wrapping the extensions around retracted skin tissue.

14. The surgical site cover of claim 1 wherein the surgical site cover further comprises non-adhesive extensions suitable for wrapping the extensions around retracted skin tissue.

15. The surgical site cover of claim 1 wherein the adhesive coated film has a stretch release strength of no greater than 1.34 kgf/19 inch for elongations ranging from 60% to 240%.

16. The surgical site cover of claim 1 wherein the surgical site cover is a surgical eye cover.

17. A surgical site cover kit comprising
an adhesive coated film comprising
a pressure sensitive skin contact adhesive layer disposed on a major surface of a conformable polymeric film layer;
an opening of suitable size to expose a surgical site;
optionally a permanent or removable carrier frame at the periphery of the surgical site cover;
at least one retraction member comprising a first end portion comprising a first attachment member for attachment to the adhesive coated film and a second attachment member at an opposing end portion;
wherein the conformable polymeric film is characterized by any one or combination of the following properties:
i) a F₁₀ value of less than 1000 grams force/inch;
ii) a Handle-O-Meter value less than 5 grams force/inch;
iii) a maximum elongation greater than 200%; and
iv) a tensile strength of no greater than 1 kgf/inch (2.54 cm) for an elongation of 5% or 10%.

18. (canceled)

19. (canceled)

20. A method of applying a surgical site cover comprising:
providing a surgical site cover of claim 1; and
adhering the pressure sensitive skin contact adhesive layer to skin tissue such that the opening or peripheral edge is adjacent the surgical site.

21. The method of claim 20 wherein when the surgical cover kit is provided, the retraction member is attached to the adhesive coated film proximate the opening or peripheral edge either prior to or after adhering the pressure sensitive skin contact adhesive layer to the skin tissue.

22. The method of claim 20 further comprising retracting skin tissue adjacent the surgical site by meaning of applying a force to the retraction members.

23. The method of claim 22 further comprising maintaining the retraction by securing the attachment member of the retraction member.

24.-28. (canceled)
